(19) United States
(12) Patent Application Publication
(10) Pub. No.: US 2012/0006055 A1
(43) Pub. Date: Jan. 12, 2012

(54) PROCESS AND APPARATUS FOR SEPARATING A GASEOUS PRODUCT FROM A FEED STREAM COMPRISING CONTAMINANTS

(76) Inventors: Helmar Van Santen, Amsterdam (NL); Cornelius Johannes Schellekens, Amsterdam (NL); Peter Veenstra, Amsterdam (NL)

(21) Appl. No.: 13/143,463

(22) PCT Filed: Jan. 6, 2010
(86) PCT No.: PCT/EP2010/050066

§ 371 Date: Sep. 14, 2011

(30) Foreign Application Priority Data
Jan. 8, 2009 (EP) 09150254.2

(51) Int. Cl. F25J 3/08; C01B 3/02; C07C 7/00; C01B 3/00; C01B 31/18

(52) U.S. Cl. 62/618; 423/648.1; 423/418.2; 585/812; 252/373; 62/617

(57) ABSTRACT

A process and apparatus for separating at least part of a gaseous product from a feed stream which comprises contaminants. The process comprises:
1) providing the feed stream;
2) cooling the feed stream to a temperature at which a slurry stream is formed which comprises solid contaminant, liquid phase contaminant and the gaseous product;
3) introducing the slurry stream as obtained in step 2) via a plurality of tangentially directed inlet means into an upper part of a separation device, thereby creating a swirl of the slurry stream which allows at least part of the gaseous product to flow upwardly and solid contaminant and liquid phase contaminant to flow downwardly;
4) removing at least part of the gaseous product from the upper part of the device; and
5) removing a stream comprising liquid phase contaminant from a lower part of the device.

The present invention concerns a process for separating a gaseous product from a feed stream which comprises contaminants, a cryogenic separation device to carry out the process, and products made in the process.

Gas streams produced from subsurface reservoirs such as natural gas, associated gas and coal bed gas methane, or from (partial) oxidation processes such as syngas and fluegas, usually contain in addition to the gaseous product concerned such as methane, hydrogen, nitrogen and/or carbon monoxide contaminants like carbon dioxide, hydrogen sulphide, carbon oxysulphide, mercaptans, sulphides and aromatic sulphur containing compounds in varying amounts. For most of the applications of these gaseous products, the contaminants need to be removed either partly or almost completely, depending on the specific contaminant and/or the use. Often, the sulphur compounds need to be removed into the ppm level, carbon dioxide sometimes up till ppm level, e.g. LNG applications, or up till 2 or 3 vol. percent, e.g. for use as heating gas. Higher hydrocarbons may be present, which, depending on the use, may be recovered. The removal of acid contaminants, especially carbon dioxide and/or hydrogen sulphide, from methane containing gas streams has been described in a number of publications.

In WO 03/062725, a process is described for the removal of freezable species from a natural gas stream by cooling a natural gas stream to form a slurry of solid acidic contaminants in compressed liquefied natural gas. The solids are separated from the liquid by means of a cyclone. It will be clear that a complete separation of the liquid from the solids is not easily achieved.

In U.S. Pat. No. 4,533,372, a cryogenic process is described for the removal of carbon dioxide and other acidic gases from methane-containing gas by treating the feed stream in a distillation zone and a controlled freezing zone. This is a rather complicated process requiring very specific equipment.

In U.S. Pat. No. 3,398,544, the removal of acid contaminants from a natural gas stream is described by cooling to liquefy the stream and to partly solidify the stream, followed by expansion and separation of cleaned gas and liquid streams from the solids. Solid contaminants need to be removed from the separation vessel, which is a complicated process when the loss of natural gas liquid is to be minimized.

In WO 2004/070297, a process for removing contaminants from a natural gas stream has been described. In a first step, water is removed from the feed gas stream. This is especially done by cooling the feed gas stream resulting in methane hydrate formation, followed by removal of the hydrates. Further cooling results in the formation of solid acidic contaminants. After separation of the solid acidic contaminants a cleaned natural gas stream is obtained. It is preferred to convert the solid contaminant into a liquid by heating the solids.

Object of the present invention is to provide an improved cryogenic process for separating a gaseous product from a feed stream that comprises contaminants.

Surprisingly, it has now been found that this can be established when the feed stream is cooled into a slurry stream which comprises solid contaminant, liquid phase contaminant and the gaseous product, and the slurry stream so obtained is introduced into a separation device by means of a particular set of inlet means.

Accordingly, the present invention relates to a process for separating at least part of a gaseous product from a feed stream which comprises contaminants, the process comprising:
1) providing the feed stream;
2) cooling the feed stream to a temperature at which a slurry stream is formed which comprises solid contaminant, liquid phase contaminant and the gaseous product;
3) introducing the slurry stream as obtained in step 2) via a plurality of tangentially directed inlet means, with a small inlet angle, into an upper part of a separation device, thereby creating a swirl of the slurry stream which allows at least part of the gaseous product to flow upwardly and solid contaminant and liquid phase contaminant to flow downwardly;
4) removing at least part of the gaseous product from the upper part of the device; and
5) removing a stream comprising liquid phase contaminant from a lower part of the vessel.

The use of the plurality of inlet means brings about a highly effective separation of gaseous product from a feed stream that comprises contaminants. Moreover, risk of ice formation on the inner wall of the separation device and erosion of the inner wall can attractively be reduced when compared with the use of a conventional cyclonic inlet.

In the process of the present invention, the feed stream in step 1) suitably has a temperature between −20 and 150° C., preferably between −10 and 70° C., and a pressure between 10 and 150 bara, preferably between 80 and 120 bara.

The slurry stream as obtained in step 2) suitably has a temperature between −40 and −100° C., preferably between −50 and −80° C.

The feed stream is suitably a hydrocarbonaceous stream or a product stream as obtained from a partial or complete oxidation process.

Suitably, the hydrocarbonaceous stream to be used in accordance with the present invention is a natural gas stream in which the gaseous contaminants are carbon dioxide and/or hydrogen sulphide and/or C2+-hydrocarbons.

Natural gas streams may become available at a temperature of from −5 to 150° C. and a pressure of from 10 to 700 bar, suitably from 20 to 200 bar.

The amount of the hydrocarbon fraction in such a feed gas stream is suitably from 10 to 85 mol % of the gas stream, preferably from 25 to 80 mol %. The hydrocarbon fraction of the natural gas stream comprises especially at least 75 mol % of methane, preferably 90 mol %. Hence, the gaseous product suitably comprises methane.

The hydrocarbon fraction in the natural gas stream may suitably contain from 0 to 20 mol %, suitably from 0.1 to 10 mol %, of C₂-C₆ compounds. The gas stream may also comprise up to 20 mol %, suitably from 0.1 to 10 vol % of nitrogen, based on the total gas stream.

Suitable examples of product streams as obtained from a partial or complete oxidation processes include fluegas and syngas which contain as gaseous products hydrogen, nitrogen and/or carbon monoxide. Therefore, in accordance with the present invention the gaseous product can also comprise hydrogen, nitrogen and/or carbon monoxide.

Suitably, the solid contaminant to be formed in step 2) comprises carbon dioxide and the liquid phase contaminant comprises hydrogen sulfide. The amount of carbon dioxide in the gas stream is suitably from 5 to 90 vol %, preferably from 10 to 75 vol %, and/or the amount of hydrogen sulphide in the gas stream is suitably from 5 to 40 vol % of the gas stream, preferably from 20 to 35 vol %. Basis for these amounts is the total volume of hydrocarbons, hydrogen sulphide and carbon dioxide. It is observed that the present process is especially suitable for feed streams comprising large amounts of sour contaminants, e.g. 10 vol % or more, suitably from 15 to 90 vol %.

The feed stream may be pre-treated for partial or complete removal of water and optionally some heavy hydrocarbons. This can for instance be done by means of a pre-cooling cycle, against an external cooling loop, a cold internal process stream, or a cold LNG stream. Water may also be removed by means of pre-treatment with molecular sieves, e.g. zeolites, aluminium oxide or silica gel or other drying agents. Water may also be removed by means washing with glycol, MEG, DEG or TEG, or glycerol. Other processes for forming methane hydrates or for drying natural gas are also possible. Water may also be removed by hydrate formation in the way as described in WO2004/070297. Suitably, water is removed until the amount of water in the natural gas stream comprises at most 50 ppmw, preferably at most 20 ppmw, more preferably at most 1 ppmw of water, based on the total feed gas stream.

In step 2, the feed stream is cooled. The cooling of the feed gas stream is suitably done by heat exchange against a cold fluidum, especially an external refrigerant, e.g. a propane cycle, an ethane/propane cascade or a mixed refrigerant cycle, or an internal process loop, suitably a carbon dioxide or hydrogen sulphide stream, a cold methane enriched stream or a cold LNG stream.

In a preferred embodiment, the liquid phase contaminant obtained in step 5, optionally after liquefaction, may be used as an internal cooling stream.

In a preferred embodiment, additional cooling of the feed stream is done by nearly isentropic expansion of the feed stream, especially by means of an expander, preferably a turbo expander or laval nozzle. In another preferred embodiment, additional cooling of the feed stream is done by isenthalpic expansion, preferably isenthalpic expansion over an orifice or a valve, especially over a Joule-Thomson valve.

Suitably the pressure drop in the expansion step is between 15 and 80 bar, preferably between 25 and 45 bar.

Preferably, the cooling stage of the feed stream comprises one or more expansion steps. For this purpose conventional equipment may be used. Conventional equipment includes turbo-expanders, so-called Joule-Thomson valves and venturi tubes. It is preferred to at least partly cool the gas stream over a turbo-expander, releasing energy. One advantageous effect of using the turbo-expander is that the energy that is released in the turbo-expander can suitably be used for compressing at least part of the gaseous product that is obtained after the contaminants are removed. Since the stream of the gaseous product is smaller than the feed gas stream now that contaminants have been removed, the energy is suitably such that the gaseous product may be compressed to an elevated pressure that makes it suitable for transport in a pipeline. In the process according to the present invention use is made of a plurality of tangentially directed inlet means, with a small inlet angle, for introducing the slurry stream as obtained in step 2) into the upper part of the separation device.

In accordance with the present invention use is made of two or more inlet means. Preferably, use is made of 2-8 inlet means, more preferably 2-6 inlet means.

In a preferred embodiment of the present invention use is made of at least two inlet means which are located at substantially the same horizontal level at circumferential spaced points. The term “substantially” is meant to indicate here that embodiments wherein the inlet means are located at levels that slightly deviate from the same horizontal level are also part of the present invention. Preferably, the at least two inlet means are located at the same horizontal level at circumferential spaced points.

In a particular attractive embodiment of the present process use is made of four inlet means which are located at substantially the same horizontal level at circumferential spaced points. Preferably, the four inlet means are located at the same horizontal level at circumferential spaced points.

In accordance with the present invention, the plurality of outlet means have a small inlet angle. The term “inlet angle” is in the context of the present invention defined as the angle between the symmetry axis of each inlet means and the line through the centre of each inlet means and the centre of the separation device at substantially the same horizontal level. The inlet angle may suitable range from 0.5 to 45 degrees. Preferably, the inlet angle is between 0.5-10 degrees, more preferably between 1-8 degrees, and most preferably between 3-5 degrees.

Preferably, the inlet angles of the plurality of inlet means are all orientated in the same direction.

Suitably, the feed stream is pre-cooled to a temperature between 15 and −45° C., preferably between 5 and −25° C., before the cooling in step 2) takes place

Suitably, such a pre-cooling of the feed gas stream is done by heat exchange against a cold fluidum, especially an external refrigerant, e.g. a propane cycle, an ethane/propane cascade or a mixed refrigerant cycle, or an internal process loop, suitably a carbon dioxide of hydrogen sulphide stream, or a cold methane stream.

The stream comprising liquid phase contaminant which is to be removed in step 5) from a lower part of the device can suitably be a slurry of solid contaminant and liquid phase contaminant.

At least part of such a slurry of solid contaminant and liquid phase contaminant as removed in step 5) can suitably be passed to a device, preferably a heat exchanger, wherein at least part of the solid contaminant present in the slurry is melted and at least part of the liquid phase contaminant so obtained can suitably be recycled to the top or intermediate part of the separation device. Preferably, at least 95% of the solid contaminant present in the slurry is melted, more preferably at least 98%. Most preferably, all the solid contaminant present in the slurry of contaminants is melted in the heat exchanger.

Suitably, between 1 and 90 vol % of the liquid phase contaminant obtained in the heat exchanger is recycled to the top or intermediate part of the separation device, preferably between 5 and 80 vol % of the liquid phase contaminant as obtained in the heat exchanger. It is also possible to recycle all liquid phase contaminant as obtained in the heat exchanger to the top or intermediate part of the separation device.

Preferably, a stream comprising at least part of the liquid phase contaminant as obtained in the heat exchanger is introduced into the top or intermediate part of the separation device which stream is introduced at a level below the location where the plurality of inlet means is arranged. In this way, the slurry stream can be diluted and the diluted slurry stream so obtained can be introduced via a slurry pump, preferably an eductor, into the heat exchanger in which heat exchanger solid contaminant present in the diluted slurry of contaminants is melted into liquid phase contaminant, whereby the diluted slurry is used as a suction fluid for the eductor, the heat exchanger is positioned outside the separation device, and the eductor is arranged inside or outside the separation device or partly inside and outside the separation device. Depending on the process conditions the stream of liquid phase to be recycled to the top or intermediate part of the separation device can also be used to strip some hydrocarbons and/or pre-melt some of the solids in the slurry stream of contaminants which has been introduced into the separation device in step 3). Suitably, from the separation device, especially from a bottom part of the separation device, a stream of liquid phase contaminant is removed.

In the event that the stream comprising liquid phase contaminants mainly comprises carbon dioxide and is therefore a CO2-rich stream, preferably the CO2-rich stream is further pressurised and injected into a subterranean formation, preferably for use in enhanced oil recovery or for storage into an aquifer reservoir or for storage into an empty oil reservoir. It is an advantage that a liquid CO2-rich stream is obtained, as this liquid stream requires less compression equipment to be injected into a subterranean formation.

Optionally, the stream of liquid contaminant o is separated into a liquid product stream and a recirculation stream which is used as a motive fluid in the eductor in the case that an eductor is used. Suitably, between 25 and 95 vol % of the stream of liquid phase contaminant that is removed from the bottom part of the separation device can be used as a motive fluid in the eductor, preferably between 30 and 85 vol % of the stream of liquid phase contaminant removed from the bottom part of the separation device, in case an eductor is used.

In accordance with the present invention a continuously moving slurry phase can be obtained, minimizing the risk of any blockages in the cryogenic separation device or in the pipelines and other pieces of equipment. Further, when a fully liquid stream is withdrawn from the heat exchanger, the absence of solid contaminant reduces the risk of blockages or erosion in subsequent pipelines or other equipment, and no damages will occur to any devices having moving parts, such as pumps. Moreover, when a pure liquid stream is withdrawn from the heat exchanger, a relatively cold liquid stream is obtained, thus minimizing the heat requirements of the separation device, and maintaining a high amount of exchangeable cold in the product stream.

It is observed that the liquid phase contaminant to be recycled to the separation device may contain some vapour and/or flash gas, e.g. up till 10 wt %, especially up till 5 wt %, more especially up till 2 wt %, of the total liquid phase contaminant.

In accordance with the present invention the heat exchanger is preferably arranged at a level positioned below the level at which the slurry pump, preferably the eductor, is arranged.

In accordance with the present invention, preferably, use is made of an eductor for removing the diluted slurry of contaminants from the separation device and passing/introducing said slurry into the heat exchanger. The diluted slurry of contaminants functions as the suction fluid in the eductor, whereas the recirculation stream to be introduced in the eductor functions as the motive fluid, in case an eductor is used.

Eductors are as such well-known and have extensively been described in the prior art. In accordance with the present invention any type of eductor can be used. Also a configuration may be used in which multiple eductors are uses.

The eductor to be used in accordance with the present invention is preferably a liquid jet solid pump.

Preferably, the eductor is arranged inside the separation device or partly inside and outside the separation device, usually a vessel.

Suitably, a housing can be positioned around the eductor, enabling the eductor to be removed from the separation device. Such a housing can, for instance, be a vessel like containment, e.g. a pipe, that can be isolated from the process through valves.

The eductor can be of such a size that it fits completely in the separation device or it may cover the entire diameter of the separation device, usually a vessel. However, it may also extend at two locations through the internal wall of the separation device.

The liquid phase contaminant to be used as the motive fluid is preferably introduced into the bottom part of the separation device at a level which is higher than the level at which the liquid phase contaminant is removed from the bottom part of the separation device. As a result free flash gas and/or vapour can escape to the top part of the cryogenic separation device.

In general, the gaseous product is removed from the top part of the cryogenic separation device at a high level, preferably at the top of the separation device.

The outlet for the gaseous product will usually be above the level at which the stream of liquid phase contaminant obtained from the heat exchanger is introduced into the separation device. In this way a washing stream can be created over the inside walls of the device.

The introduction of the slurry stream in step 2) will be at a level which is preferably higher than the level at which the stream of liquid phase contaminant obtained from the heat exchanger is introduced into the separation device.

Preferably, the level at which the slurry stream comprising the solid contaminant, liquid phase contaminant and the gaseous product is introduced into the separation device in step 3) will be higher than the level at which the heat exchanger will be arranged.

Preferably, the slurry pump, preferably an eductor, is arranged at a level which is higher than the level at which the heat exchanger is arranged, allowing the diluted slurry of contaminants to flow downstream into the heat exchanger.

It will be understood that the slurry pump, preferably an eductor, is arranged below the slurry level which is maintained in the separation device.

Eductors, also referred to as siphons, exhausters, eductors or jet pumps, are as such well-known and have extensively been described in the prior art. Reference herein to an eductor is to a device to pump produced solid and liquid CO2 slurry from the separator to the heat exchanger. The eductor is suitably designed for use in operations in which the head pumped against is low and is less than the head of the fluid used for pumping. For a description of suitable eductors, also referred to as eductors or jet pumps, reference is made to Perry's Handbook for Chemical Engineering, 8th edition, chapter 10.2. In accordance with the present invention any type of eductor can be used. The eductor is preferably a liquid jet solid pump.

Preferably, the eductor is arranged inside the separation device or partly inside and outside the separation device.

Suitably, a housing can be positioned around the eductor, enabling the eductor to be removed from the separation device. Such a housing can, for instance, be a vessel like containment, e.g. a pipe, that can be isolated from the process through valves.

In another embodiment of the present invention the eductor is arranged outside the separation device. Such an embodiment can be useful in situations in which the eductor in use needs to be repaired or replaced.

The eductor can be of such a size that it fits completely in the separation device or it may cover the entire diameter of the separation device, usually a vessel. However, it may also extend at two locations through the internal wall of the separation device.

The stream of liquid phase contaminant stream that is removed from the bottom part of the separation device is suitably removed at a level below the slurry level inside the separation device.

Suitable internals may be used to prevent ingress of solid particles into the withdrawal line. Preferably a pump is installed in the withdrawal line to remove the stream of liquid phase contaminant from the bottom part of the separation device, and to power the stream of liquid phase contaminant that is to be used as the motive fluid in the eductor in case an eductor is used.

In accordance with the present invention the cooling process as described in step (2) of the present process can suitably be carried out at a close distance, e.g. up to a few meters, preferably at most 1 m, to the separator device. Hence, an expansion device for carrying out step 2) can be arranged upstream of the inlet means. The inlet means may also comprise such an expansion device. In that case the inlet as such will encompass such an expansion device, minimizing any problems due to the transport of the solid particles. The separation device is suitably a vessel which comprises a vertical cylindrical housing. The diameter may vary from 1 to 10 meter, or even more, the height may vary from 3 to 35 meters or even more. In general, the slurry level in the separation vessel will vary between 30 and 70% of the height of the vessel.

The heat exchanger preferably uses a process stream to supply the heat for melting the solid contaminants. A suitable process stream is the gaseous product as removed in step 4).

Preferably, means are positioned in the separation device to direct the diluted slurry of contaminants towards the eductor in case an eductor is used. Preferably, use is made of a funnel to establish this. One or more funnels can be arranged on top of each other. Preferably in the wider part of the funnel, a grid is present to stop large chunks of falling in the more narrow inlet of the eductor/pump and in doing so, avoid plugging of the slurry pump/eductor.

The diluted slurry of contaminants can suitably be passed directly from the eductor into the heat exchanger.

In another embodiment, however, the diluted slurry of contaminants may be passed first through means such as a conduit before it is introduced into the heat exchanger. In that case the separation device also comprises means to introduce the diluted slurry of contaminants via the eductor into the heat exchanger.

In another embodiment of the present invention the heat exchanger is arranged inside the separation device, in the intermediate or bottom part of the separation device, preferably in the bottom part of the separation device. In the heat exchanger solid contaminant will at least partly be melted into liquid phase contaminant. In that case, the eductor or another type of pump, is situated below the heat exchanger and arranged outside or inside the separation device or partly inside or outside the separation device. Suitably, the eductor or other type of pump is arranged outside the separation device and communicates with the separation device. Preferably, the pump, preferably an eductor, is arranged below the separation device, more preferably, below the central bottom part of the separation device. Such an embodiment can be useful in situations in which the eductor in use needs to be repaired or replaced.

The feed stream provided in step 1) of the present process can suitably have been subjected to one or more purification processes in which gaseous contaminants are removed from the feed stream, before step 2) of the present process is carried out.

Such a purification process can suitably comprise the steps of:
a) providing a feed stream;
b) cooling the feed stream to a temperature at which liquid phase contaminant is formed as well as a gaseous phase; and
c) separating the two phases obtained in step b) by means of a gas/liquid separator, and
c) recovering the liquid phase which will be provides as the feed stream in step 1) of the present process.

Suitably, steps a)-c) can be repeated twice or three times before step 2) in accordance with the present invention is carried out. Such a process has, for instance been described in WO 2006/087332 which is hereby incorporated by reference. Hence, the feed gas stream can be subjected to a number of combinations of subsequent cooling and separation steps, before step 2) of the present invention is carried out.

Suitably, after step a) the gaseous phase can be recompressed in one or more compression steps before step 2) in accordance with the present invention is carried out. In another embodiment of the present invention the feed stream may between steps 1) and 2) be cooled to a temperature at which at least part of the feed stream is present in the liquid phase, the cooled feed stream so obtained may be separated by means of a cryogenic distillation into a bottom stream rich in liquid phase contaminant and lean in gaseous product and into a top stream rich in gaseous product and lean in gaseous contaminant, and the feed stream so obtained may then be subjected to the remaining steps 2)-5) of the process according to the present invention.

The cryogenic distillation section to be used in the cryogenic distillation is as such known in the art.

Suitably, the feed stream is cooled to a temperature between −10 and −50° C., preferably between −20 and −40° C. before introduction into the cryogenic distillation section.

Suitably, the bottom temperature of the cryogenic distillation section is between −15 and 35° C., preferably between −5 and 30° C. A reboiler may be present to supply heat to the column.

Suitably, the top temperature of the cryogenic distillation section is between −70 and −40° C., preferably between −60 and −30° C. In the top of the cryogenic distillation column a condenser may be present, to introduce cold into the column.

In order to reach gas line specifications or LNG specifications in case methane is the gaseous product, the methane enriched gaseous phase may further be purified, in an additional cryogenic distillation process using a cryogenic distillation section which is as such known in the art.

Suitably, in such an additional cryogenic distillation process the bottom temperature of the cryogenic distillation section is between −30 and 10° C., preferably between −10 and 5° C. A reboiler may be present to supply heat to the distillation section.

Suitably, the top temperature of the cryogenic distillation section is between −110 and −80° C., preferably between −100 and −90° C. In the top of the cryogenic distillation section a condenser may be present, to provide reflux and a liquefied (LNG) product.

As an alternative, further purification of the gaseous product may be accomplished by absorption with a suitable absorption liquid. Suitable absorbing liquids may comprise chemical solvents or physical solvents or mixtures thereof.

A preferred absorbing liquid comprises a chemical solvent and/or a physical solvent, suitably as an aqueous solution.

Suitable chemical solvents are primary, secondary and/or tertiary amines, including sterically hindered amines.

A preferred chemical solvent comprises a secondary or tertiary amine, preferably an amine compound derived from ethanolamine, more especially DIPA, DEA, MMEA (monomethyl-ethanolamine), MDEA (methyldiethanolamine) TEA (triethanolamine), or DEMEA (diethyl-monoethanolamine), preferably DIPA or MDEA. It is believed that these chemical solvents react with acidic compounds such as CO2 and H2S.

Suitable physical solvents include tetramethylene sulphone (sulpholane) and derivatives, amides of aliphatic carboxylic acids, N-alkyl pyrrolidone, in particular N-methylpyrrolidine, N-alkyl piperidones, in particular N-methyl piperidone, methanol, ethanol, ethylene glycol, polyethylene glycols, mono- or di(C1-C4) alkyl ethers of ethylene glycol or polyethylene glycols, suitably having a molecular weight from 50 to 800, and mixtures thereof. The preferred physical solvent is sulfolane. It is believed that CO2 and/or H2S are taken up in the physical solvent and thereby removed.

Other treatments of the gaseous product may include a further compression, when the gaseous product is wanted at a higher pressure. If the amounts of contaminants in the gaseous product are undesirably high, the gaseous product may be subjected to one or more repetitions of the present process.

The invention further provides purified gaseous product obtained by the process.

In one advantageous application, the process enables purification of natural gas comprising substantial amounts of acidic contaminants, such as carbon dioxide, resulting in purified natural gas comprising low levels of contaminants, especially of sulphur contaminants. The production of LNG from such natural gas, which would be very difficult if not impossible by conventional processes, is made possible.

Thus, the invention also provides LNG obtained from liquefying purified natural gas obtained by the process. The LNG thus-obtained typically has very low concentrations of contaminants other than natural gas.

The present invention also relates to a cryogenic separation device for carrying out the process of the present invention.

Accordingly, the present invention also provides a cryogenic separation device for carrying out the present process, the device having outlet means for removing at least part of the gaseous product from an upper part of the device, outlet means for removing the stream comprising liquid phase contaminant from a lower part of the device, a plurality of tangentially directed inlet means for introducing the slurry stream comprising solid contaminant, liquid phase contaminant and the gaseous product into an upper part of the vessel, with a small inlet angle, whereby the inlet means are arranged below the outlet means for removing at least part of the gaseous product from the device, and each inlet means comprises expansion means or communicates with expansion means arranged upstream of the inlet means.

Preferably, the separation device comprises two or more inlet means, preferably 2-8 inlet means.

Preferably, at least two inlet means are located at substantially the same horizontal level at circumferential spaced points.

In a particularly attractive embodiment, the separation device comprises 4 inlet means which are located at substantially the same horizontal level at circumferential spaced point.

Suitably, the inlet angle is between 0.5-45 degrees, preferably between 0.5-10 degrees, mote preferably between 1-8 degrees, and most preferably between 3-5 degrees, wherein the terms “inlet angle” and “substantially” have been defined hereinabove.

Preferably, the inlet angles are all orientated in the same direction.

Suitably, the cryogenic separation device according to the present invention further comprises a heat exchanger arranged outside the device, an eductor arranged inside or outside the device or partly inside and outside the device at a level below the plurality of inlet means, wherein either the outlet means for removing the slurry from the lower part of the device communicates with the eductor and the eductor communicates in turn with the heat exchanger or the eductor communicates with the outlet means for removing the slurry from the lower part of the device and said outlet means communicate in turn with the heat exchanger, and the heat exchanger communicates with means for recycling at least part of the liquid phase contaminant to a lower part of the device.

Suitably, the expansion means comprises an orifice or a valve, especially a Joule-Thomson valve, or an expander, preferably a turbo expander or a laval nozzle.

The present invention also relates to a cryogenic separation device for carrying out the process according to present invention, which separation device comprises a top, intermediate and bottom part; a plurality of inlet means as defined hereinbefore to introduce a slurry stream which comprises solid contaminant, liquid phase contaminant and a gaseous product into the top or intermediate part of the separation device; means to remove at least part of the gaseous product from the top part of the separation device; means for introducing a stream comprising liquid phase contaminant into the top or intermediate part of the separation device to dilute the slurry of contaminants inside the separation device; a heat exchanger arranged outside the separation device; a slurry pump, preferably an eductor, arranged inside or outside the separation device or partly inside and outside the separation device at a level that is below the level at which the means for introducing the slurry of contaminants into the separation device is arranged, which eductor communicates with the heat exchanger; means for directing the diluted slurry of contaminants inside the separation device towards the eductor; means to introduce liquid phase contaminant obtained in the heat exchanger to the bottom part of the separation device; means to introduce liquid phase contaminant obtained in the heat exchanger into the top or intermediate part of the separation device; means to remove liquid phase contaminant from the bottom part of the separation device; means to separate liquid phase contaminant removed from the bottom part into a liquid product stream and a recirculation stream for use as a motive fluid in the eductor in the case an eductor is used.

The means for directing the diluted slurry of contaminants inside the separation device towards the slurry pump, especially the eductor, can suitably comprise a funnel. Suitably, use can be made of a number, for instance two, funnels that are arranged one above the other.

The present invention further relates to a cryogenic separation device which comprises a top part, an intermediate part and a bottom part; a plurality of inlet means as defined hereinbefore to introduce a slurry stream which comprises solid contaminant, liquid phase contaminant and a gaseous product into the top or intermediate part of the separation device; means to remove at least part of the gaseous product from the top part of the separation device; means for introducing a stream comprising liquid phase contaminant into the top or intermediate part of the separation device to dilute the slurry inside the separation device; a heat exchanger arranged inside the separation device; a pump, preferably an eductor, which is arranged inside or outside the separation device or partly inside and outside the separation device at a level which is below the level at which the heat exchanger is arranged for removing a stream comprising liquid phase contaminant from the separator; means to remove a stream comprising liquid phase contaminant from the intermediate or bottom part of the separation device; and means to separate liquid phase contaminant removed from the intermediate or bottom part into a liquid product stream and a recirculation stream for use as a motive fluid in the eductor in the case an eductor is used.

Preferably, the heat exchanger is arranged in the bottom part of the separation device.

Preferably, the pump, preferably an eductor is arranged outside the separation device and communicates with the separation device. Preferably, the pump, preferably an eductor, is arranged below the separation device, more preferably below the central bottom part of the separation device.

In general, the top part of the of the separation device will comprise the top quarter length of the device, whereas the bottom part will comprise the bottom quarter length of the device. The intermediate part will comprise the remaining.

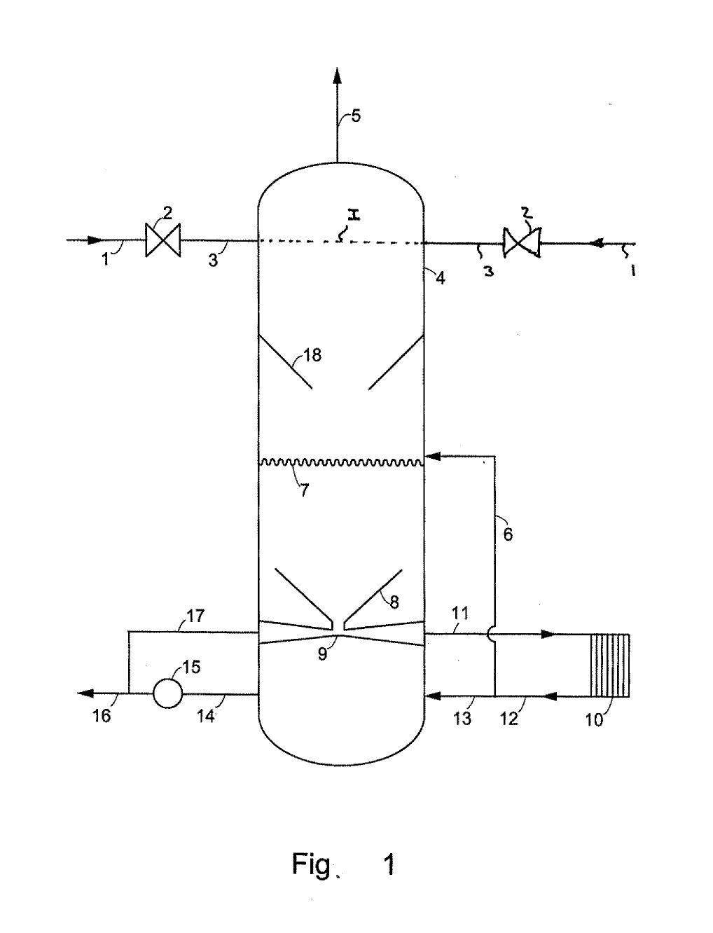

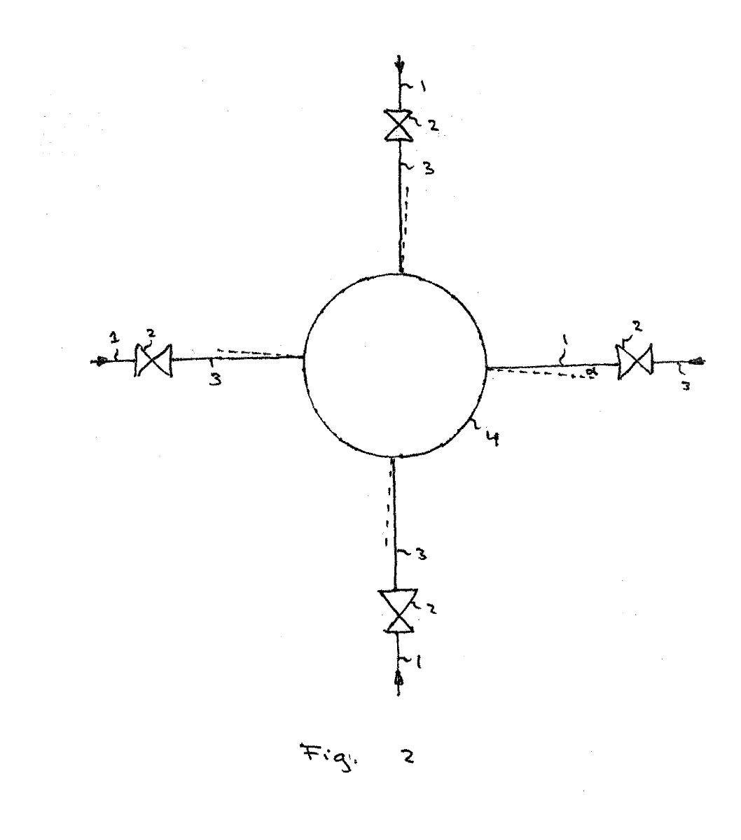

The invention will be further illustrated by means of FIGS. 1 and 2. Figure represents schematically a longitudinal section of the cryogenic separation device according to the present invention, whereas FIG. 2 represents schematically a cross-section of said separation device along the dotted line I. In FIG. 1, a natural gas is passed via four conduits 1 (of which only two conduits are shown) through expansion means 2, especially Joule Thomson valves, whereby four streams are obtained of slurries which comprises solid contaminant, liquid phase contaminant and a methane enriched gaseous phase. The slurry streams flow via conduits 3 (only two are shown) into cryogenic separation device 4. A methane enriched gaseous is removed from the separation device via a conduit 5. A stream of liquid phase contaminant is introduced into the separation device via a conduit 6 to dilute the slurry inside the separation device, establishing or maintaining a slurry level 7. The diluted slurry of contaminated is directed by means of a funnel 8 towards the top opening of an eductor 9. In the eductor 9 the diluted slurry is used as a suction fluid and via the eductor 9 it is passed into a heat exchanger 10 via a conduit 11. In the heat exchanger 10 solid contaminant present in the diluted slurry is melted into liquid phase contaminant. Part of the liquid phase contaminant so obtained is passed via a conduit 12 to the conduit 6, whereas the main part of liquid phase contaminant is introduced into the bottom part of the separation device 4 by means of a conduit 13. Liquid phase contaminant is subsequently withdrawn from the separation vessel 4 by means of a conduit 14 using a pump 15. Part of the withdrawn liquid phase contaminant is recovered as a product stream via a conduit 16 and part of said liquid phase contaminant is recycled via a conduit 17 to the eductor 9. A funnel 18 is present to guide the slurry stream into the direction of funnel 8.

In FIG. 2, four feed stream are passed via conduits 1 through expansion means 2, after which the slurry streams so obtained are introduced by means of inlet means 3 into the separation device 4. The inlet angle (α) of the four inlet means 3 is in all cases 4 degrees, and is orientated in the same direction.

1. A process for separating at least part of a gaseous product from a feed stream which comprises contaminants, the process comprising:
1) providing the feed stream;
2) cooling the feed stream to a temperature at which a slurry stream is formed which comprises solid contaminant, liquid phase contaminant and the gaseous product;
3) introducing the slurry stream as obtained in step 2) via a plurality of tangentially directed inlet means, with a small inlet angle, into an upper part of a separation device, thereby creating a swirl of the slurry stream which allows at least part of the gaseous product to flow upwardly and solid contaminant and liquid phase contaminant to flow downwardly;
4) removing at least part of the gaseous product from the upper part of the device; and
5) removing a stream comprising liquid phase contaminant from a lower part of the device.

2. The process according to claim 1, wherein the feed stream in step 1) has a temperature between −20 and 150° C. and a pressure between 10 and 150 bara.

3. The process according to claim 1 wherein the slurry stream as obtained in step 2) has a temperature between −40 and −100° C., preferably between −50 and −80° C., and a pressure between 5 and 200 bara, preferably between 55 and 75 bara.

4. The process according to claim 1 wherein the cooling in step 2) has been established by means of isenthalpic expansion, preferably isenthalpic expansion over an orifice or a valve, especially a Joule-Thomson valve, or by means of nearly isentropic expansion, preferably by means of an expander, especially a turbo expander or a laval nozzle.

5. The process according to claim 1 in which at least part of the slurry of the solid contaminant and liquid phase contaminant as removed in step 5) is passed to a heat exchanger wherein substantially all the solid contaminant present in the slurry stream is melted and at least part of the liquid phase contaminant so obtained is recycled to a lower part of the device.

6. The process according to claim 1 wherein the feed stream is a hydrocarbonaceous stream or a product stream as obtained from a partical or complete oxidation process.

7. The process according to claim 1 wherein the gaseous product comprises methane or hydrogen and/or carbon monoxide.

8. The process according to claim 1 wherein the solid contaminant comprises carbon dioxide and the liquid phase contaminant comprises hydrogen sulfide.

9. The process according to claim 1 wherein use is made of two or more inlet means, preferably 2-8 inlet means, preferably wherein at least two inlet means are located at substantially the same horizontal level at circumferential spaced points.

10. The process according to claim 1 wherein use is made of four inlet means which are located at substantially the same horizontal level at circumferential spaced points, preferably wherein the inlet angle is between 0.5-45 degrees, more preferably wherein the inlet angles are all orientated in the same direction.

11. The cryogenic separation device for carrying out the process according to claim 1 the device having outlet means for removing at least part of the gaseous product from an upper part of the device, outlet means for removing the stream comprising liquid phase contaminant from a lower part of the device, a plurality of tangentially directed inlet means for introducing the slurry stream comprising solid contaminant, liquid phase contaminant and the gaseous product into an upper part of the device, with a small inlet angle, whereby the inlet means are arranged below the outlet means for removing at least part of the gaseous product from the device, and each inlet means comprises expansion means or communicates with expansion means arranged upstream of the inlet means.

12. The device according to claim 11, in which the inlet angle is between 0.5-45 degrees, preferable in which the inlet angles are all orientated in the same direction.

13. The device according to claim 11 wherein the device further comprises a heat exchanger arranged outside the device, an eductor arranged inside or outside the device or partly inside and outside the device at a level below the plurality of inlet means, wherein either the outlet means for removing the slurry from the lower part of the device communicates with the eductor and the eductor communicates in turn with the heat exchanger or the eductor communicates with the outlet means for removing the slurry from the lower part of the device and said outlet means communicate in turn with the heat exchanger, and the heat exchanger communicates with means for recycling at least part of the liquid phase contaminant to a lower part of the device or a device according to claim 11, wherein the expansion means comprises an orifice or a valve, especially a Joule-Thomson valve, or an expander, preferably a turbo expander or a laval nozzle.

14. The Purified stream containing at least part of the gaseous product obtained by a process according to claim 1.

15. The process for liquefying a feed stream comprising purifying the feed stream according to claim 1 followed by liquifying the feed stream by methods known in the art.

16. The cryogenic separation device for carrying out the process according to claim 1 wherein the inlet means comprises two or more inlet means

17. The cryogenic separation device for carrying out the process according to claim 16 wherein the inlet means comprises 2-8 inlet means.

18. The cryogenic separation device for carrying out the process according to claim 16 wherein at least two inlet means are located at substantially the same horizontal level at circumferential spaced points.

19. The cryogenic separation device for carrying out the process according to claim 16 wherein the inlet means comprises 4 inlet means which are located at substantially the same horizontal level at circumferential spaced points
